SEQUENCE LISTING (26 sequences)

SEQ ID NO: 1 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 493 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGFSDKDLEDIVGGGEYKPDKGKGDGRYGSNDDPGSGMVAEPGTIAGVASALAMALIGAVSSYISYQQKKFCFSIQKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 537 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGFSDKDLEDIVGGGEYKPDKGKGDGRYGSNDDPGSGMVAEPGTIAGVASALAMALIGAVSSYISYQQKKFCFSIQQGLNADYVKGENLEAVVCEEPQVKYSTLHTQSAEPPPPPEPARIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 1461 bp, synthetic construct
  source 1..1461: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 5 - DNA, 1482 bp, synthetic construct
  source 1..1482: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaggcggcttcagcgacaaggacctggaagatatcgttggcggcggagagtacaagcccgacaaaggcaaaggcgacggcagatacggcagcaacgatgatcctggctctggcatggtggccgagcctggaacaattgctggcgtggcatctgccctggccatggctcttattggagccgtgtccagctacatcagctaccagcagaagaagttctgcttcagcatccagaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcccgcttatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 6 - DNA, 1614 bp, synthetic construct
  source 1..1614: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaggcggcttcagcgacaaggacctggaagatatcgttggcggcggagagtacaagcccgacaaaggcaaaggcgacggcagatacggcagcaacgatgatcctggctctggcatggtggccgagcctggaacaattgctggcgtggcatctgccctggccatggctcttattggagccgtgtccagctacatcagctaccagcagaagaagttctgcttcagcatccagcagggcctgaacgccgattacgtgaagggcgagaatctggaagccgtcgtgtgcgaggaaccccaagtgaagtacagcaccctgcacacccagtctgccgaacctccacctcctccagaacctgccagaatcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgacgctcccgcttatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggcatgaagggcgaacgcagaagaggcaagggacacgatggcctgtatcagggcctgtctaccgccaccaaggacacctatgatgccctgcacatgcaggctctgcctccaagataa

SEQ ID NO: 7 - protein, 21 aa, synthetic construct
MALPVTALLLPLALLLHAARP

SEQ ID NO: 8 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 9 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 10 - protein, 43 aa, synthetic construct
GGFSDKDLEDIVGGGEYKPDKGKGDGRYGSNDDPGSGMVAEPG

SEQ ID NO: 11 - protein, 33 aa, synthetic construct
TIAGVASALAMALIGAVSSYISYQQKKFCFSIQ

SEQ ID NO: 12 - protein, 44 aa, synthetic construct
QGLNADYVKGENLEAVVCEEPQVKYSTLHTQSAEPPPPPEPARI

SEQ ID NO: 13 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 42 aa, synthetic construct
KRGRKKLLYIFKQPFMRPVQTTQEEDGCSCRFPEEEEGGCEL

SEQ ID NO: 16 - DNA, 63 bp, synthetic construct
  source 1..63: mol_type = other DNA
atggccttaccagtgaccgccttgctcctgccgctggccttgctgctccacgccgccaggccg

SEQ ID NO: 17 - DNA, 726 bp, synthetic construct
  source 1..726: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 18 - DNA, 207 bp, synthetic construct
  source 1..207: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 19 - DNA, 129 bp, synthetic construct
  source 1..129: mol_type = other DNA
ggcggcttcagcgacaaggacctggaagatatcgttggcggcggagagtacaagcccgacaaaggcaaaggcgacggcagatacggcagcaacgatgatcctggctctggcatggtggccgagcctgga

SEQ ID NO: 20 - DNA, 99 bp, synthetic construct
  source 1..99: mol_type = other DNA
acaattgctggcgtggcatctgccctggccatggctcttattggagccgtgtccagctacatcagctaccagcagaagaagttctgcttcagcatccag

SEQ ID NO: 21 - DNA, 132 bp, synthetic construct
  source 1..132: mol_type = other DNA
cagggcctgaacgccgattacgtgaagggcgagaatctggaagccgtcgtgtgcgaggaaccccaagtgaagtacagcaccctgcacacccagtctgccgaacctccacctcctccagaacctgccagaatc

SEQ ID NO: 22 - DNA, 339 bp, synthetic construct
  source 1..339: mol_type = other DNA
agagtgaagttctccagatccgccgacgctcccgcttatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggcatgaagggcgaacgcagaagaggcaagggacacgatggcctgtatcagggcctgtctaccgccaccaaggacacctatgatgccctgcacatgcaggctctgcctccaagataa

SEQ ID NO: 23 - DNA, 339 bp, synthetic construct
  source 1..339: mol_type = other DNA
agagtgaagttcagcagatccgccgacgctcccgcttatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 24 - DNA, 339 bp, synthetic construct
  source 1..339: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 25 - DNA, 126 bp, synthetic construct
  source 1..126: mol_type = other DNA
aagcggggcagaaagaagctgctgtacatcttcaagcagcccttcatgcggcccgtgcagaccacacaagaggaagatggctgctcctgcagattccccgaggaagaagaaggcggctgcgagctg

SEQ ID NO: 26 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

(19) United States
(12) Patent Application Publication
(10) Pub. No.: US 2025/0032541 A1
(43) Pub. Date: Jan. 30, 2025

(54) NOVEL CHIMERIC ANTIGEN RECEPTOR (CAR) HAVING ENHANCED FUNCTIONS

(71) Applicant: TICAROS CO., LTD, Seoul (KR)

(72) Inventors: Kyungho CHOI, Seoul (KR); Eun-Young CHOI, Seoul (KR); Giri NAM, Seoul (KR); Hanna CHANG, Seoul (KR); Hyeonji LIM, Seoul (KR); Hye-Ran YEON, Seoul (KR); Hyung-Bae PARK, Seoul (KR)

(73) Assignee: TICAROS CO., LTD, Seoul (KR)

(21) Appl. No.: 18/686,998

(22) PCT Filed: Aug. 26, 2022
(86) PCT No.: PCT/KR2022/012556

§ 371 Date: Oct. 16, 2024

(30) Foreign Application Priority Data
Aug. 27, 2021 (KR) 10-2021-0113648

(51) Int. Cl. A61K 35/17; A61K 39/00; A61P 35/00; C07K 16/28; C12N 15/86

(52) U.S. Cl. CPC A61K 35/17; A61K 39/4611; A61K 39/4631; A61K 39/464412; A61P 35/00; C07K 16/2803; C12N 15/86; A61K 2239/13; A61K 2239/21; A61K 2239/22; C12N 2740/15043

(57) ABSTRACT

The present invention relates to a novel chimeric antigen receptor comprising a CD99L2 region, which is known to play a key role in cell adhesion and migration, as a backbone of the chimeric antigen receptor, an immune cell comprising the same, and the uses thereof. CD99L2-based CAR-T cells exhibit enhanced T-cell activity and tumor treatment efficiency compared to conventional CAR-T cells, and thus can be useful in immune cell therapy for the treatment of cancer.

### TECHNICAL FIELD

The present invention relates to a novel chimeric antigen receptor comprising a CD99L2 region, which is known to play a key role in cell adhesion and migration, as a backbone of the chimeric antigen receptor, an immune cell comprising the same, and the uses thereof.

### BACKGROUND ART

A chimeric antigen receptor (CAR) is a fusion protein that connects a recombinant antibody (such as single-chain variable fragment (scFv)) that specifically binds to cancer antigens expressed on the surface of tumor cells to the signaling domain of a T cell receptor. CAR-T cells are transgenic T cells that artificially express a CAR on T cells isolated from a patient's blood (Kershaw MH, et al., Nat Rev Immunol. 2005;5 (12): 928-40). When the CAR protein gene is introduced to T cells in the form of retrovirus or lentivirus, more than 50% of T cells express CAR protein on the surface within 2 weeks due to high gene transfer efficiency, thereby producing a large amount of tumor-specific T cells in a short time.

When the antibody site of the CAR protein recognizes the tumor, the resulting CAR-T cells function as tumor-killing cells by sending an activation signal inside the T cells. Since the late 2000s, clinical trials of CAR-T cell therapies have surged (Jena B, et al., Blood. 2010; 116 (7): 1035-44). In particular, CAR-T cell therapy targeting CD19, a cancer antigen in B lymphocyte hematologic tumors, has shown remarkable results since early clinical trials. CD19 CAR-T cell therapy, which showed some effectiveness in B-cell lymphoma around 2010, began with the report of complete remission in patients with chronic lymphocytic leukemia refractory to conventional treatment by a research team at the University of Pennsylvania. In recent years, 27 out of 30 patients with acute lymphocytic leukemia, who were refractory to all conventional treatments, achieved complete remission with CD19 CAR-T cell therapy within one month, and the overall survival rate for 6 months was 78%, resulting in rapid growth due to large investments by a number of multinational pharmaceutical companies (Maude SL, et al., N Engl J Med. 2014; 371 (16): 1507-17). As a result, two CD19-targeted CAR-T cell therapies received FDA approval in late 2017.

Currently, CAR-T cell development is primarily focused on hematologic malignancies and is expanding to some solid tumors (Yip A, Webster RM, Nat Rev Drug Discov. 2018; 17 (3): 161-2). As a main hematologic tumor target, the development of anti-BCMA CAR-T cell therapy for multiple myeloma is the most advanced, and CAR-T cell therapies for CD20 and CD22 are being developed in addition to CD19 as B lymphatic hematologic tumor antigens. Some clinical trials have been conducted for CAR-T cell therapy for solid tumors such as GD2 (brain tumor) and Mesothelin (pleural cancer), but dramatic efficacy has not yet been reported. This is thought to be due to the presence of several factors that block the effect of CAR-T cells in solid tumors. For example, compared to leukemia, where tumor cells are mainly distributed in the blood and are less likely to create a tumor microenvironment, in solid tumors, tumor cells build an immune-resistant tumor microenvironment by secreting immunosuppressive cytokines such as TGF-beta and IL-10, recruiting immunosuppressive cells such as regulatory T cells and Myeloid-derived suppressor cells (MDSC), or expressing immunosuppressive ligands such as PD-L1 on the tumor surface (Rabinovich GA, et al., Annu Rev Immunol. 2007; 25:267-96). Therefore, for the future commercialization of CAR-T cell therapy, it is essential to develop CAR-T cells with significantly increased T cell activity that can overcome the immunosuppressive environment and exert their effects.

One strategy to enhance the function of CAR-T cells is to enhance T cell activation by altering the structure of the CAR protein itself (Dotti G, et al., Immunol Rev. 2014; 257 (1): 107-26). CAR proteins are designed in such a way that a variable region (a single chain variable fragment: scFv) of an antibody that recognizes a cancer antigen is connected to an intracellular signaling site via a backbone region (extracellular spacer+transmembrane domain). The intracellular signaling site is mainly based on the intracellular region of the CD3 zeta chain, which is a signaling subunit of the T cell receptor (1st generation CAR). To date, there have been ongoing efforts to improve the function of CAR-T cells through modification of CAR proteins, mostly by replacing or adding signaling sites on co-stimulatory molecules (Morello A, et al., Cancer Discov. 2016; 6 (2): 133-46). For example, two currently commercially available CAR-T cell therapies use the intracellular signaling domains of CD28 and 4-1BB co-stimulatory molecules, respectively (2nd generation CAR), followed by attempts for CAR simultaneously including the intracellular signaling domains of CD28 and 4-1BB (3rd generation CAR). The currently available Kymriah CAR-T cells from Novartis and Yescarta CAR-T cells from Gilead are 2nd generation CAR-T cells that use the intracellular signaling domains of 4-1BB and CD28, respectively. On the other hand, in the CAR backbone, some parts of CD8, CD28, IgG1 or lgG4 serve only as physical connections, and there are few reports in which functionality is imparted to these regions. Therefore, the replacement of CAR backbone sites may allow for novel modifications to improve the function of CAR-T cells.

Under this technical background, the present inventors investigated the possibility of improving the tumor treatment efficacy of CAR-T cells by introducing a novel CAR design that uses a transmembrane domain of the CD99L2 protein as a CAR backbone. As a result, the present invention was completed by finding that CD99L2 backbone CAR-T cells exhibit significantly improved antitumor efficacy compared to conventional CD8 backbone CAR-T cells.

The above information disclosed in the Background section is provided only for better understanding of the background of the present invention, and may not include information that constitutes prior art already known to those skilled in the art to which the present invention belongs.

### SUMMARY OF THE INVENTION

It is an object of the present invention to provide a chimeric antigen receptor, which exhibits an improved therapeutic effect against tumor, and an immune cell comprising the same.

It is another object of the present invention to provide a nucleic acid encoding the chimeric antigen receptor, an expression vector comprising the nucleic acid, and a virus comprising the expression vector.

It is still another object of the present invention to provide a composition for treating cancer comprising the immune cell, a method of treating cancer using the immune cell, the use of the immune cell for the treatment of cancer, and the use of the immune cell for the manufacture of a medicament for the treatment of cancer.

In order to achieve the above objects, the present invention provides a chimeric antigen receptor comprising a CD99L2-derived extracellular domain and a CD99L2-derived transmembrane domain.

The present invention also provides a nucleic acid encoding the chimeric antigen receptor, an expression vector comprising the nucleic acid, a virus comprising the expression vector, and an immune cell expressing the chimeric antigen receptor.

The present invention also provides a composition for treating cancer comprising the immune cell, a method of treating cancer using the immune cell, the use of the immune cell for the treatment of cancer, and the use of the immune cell for the manufacture of a medicament for the treatment of cancer.

### BRIEF DESCRIPTION OF DRAWINGS

FIG. 1 shows CD99L2-backbone-based CAR designs and in vitro activity test results. FIG. 1A schematically shows the structural designs of CAR proteins (hCD8 L: human CD8α leader, αCD19 scFv: anti-CD19 antibody (clone FMC63) single chain variable fragment, EC: extracellular region, TM: transmembrane region, cyt: cytoplasmic region). FIGS. 1B and 1E show the expression level of CAR protein on the surface of CAR-T cells (number in graph: proportion of cells (%)). FIGS. 1C and 1F are graphs showing the ability of each CAR-T cell to kill Raji-Luc lymphoma cells (relative light unit: luciferase activity value in Raji-Luc cells that survived after overnight culture with CAR-T cells, E: T ratio (effector: target ratio): cell number ratio of co-cultured CAR-T cells (effector) and Raji-Luc cells (target)). FIGS. 1D and 1G are graphs showing the amount of IFN-γ that is secreted into the supernatant after co-culture of CAR-T cells and Raji cells.

FIG. 2 indicates a flow cytometry analysis of the activation kinetics of CD99L2-backbone-based CAR-T cells, showing the time-dependent changes in the expression of CD4-positive (FIG. 2A) and CD8-positive (FIG. 2B) CAR-T cell surface activation markers upon co-culture of the CAR-T cells with Raji cells (MFI: mean fluorescent intensity).

FIG. 3 illustrates the effect of promoting tumor removal in vivo of CD99L2-backbone-based CAR-T cells, showing representative images over time obtained through bioluminescence imaging of the extent of in-vivo proliferation of tumor cells, at the time of intravenous injection of CAR-T cells on the 7^{th} day after intravenous injection of Raji-Luc cells into NSG mice (day 0).

### DETAILED DESCRIPTION AND PREFERRED EMBODIMENTS OF THE INVENTION

Unless otherwise defined, all technical and scientific terms used herein have the same meaning as commonly understood by those skilled in the art to which the present invention belongs. In general, the nomenclature used herein is well known and commonly used in the art.

A chimeric antigen receptor (CAR) is an artificial receptor that links the antigen recognition domain of an antibody with a cell membrane domain and an intracellular signaling domain. T cells (CAR-T cells) expressing this receptor by transduction have the ability to specifically kill tumors by recognizing and activating tumor surface antigens through antibody domains. Therefore, CAR-T cells have been developed as antibody gene cell therapies that combine the tumor targeting ability of antibodies and the tumor killing ability of T cells, and in particular, two or more CAR-T cell therapies have been released as they show excellent therapeutic efficacy against hematologic tumors. However, CAR-T cell therapy shows high therapeutic efficiency in hematologic tumors with a high probability of encountering tumor cells in the blood, but low efficiency for solid tumors. Therefore, for the commoditization of CAR-T cell therapy for solid tumors, the function of CAR-T cells must be improved. As part of the strategy to enhance the function of CAR-T cells, efforts are being made to produce more efficient CAR proteins by modifying the structure of CAR proteins.

The CAR backbone includes a transmembrane domain, and the transmembrane domain of a novel transmembrane protein can be utilized to enhance CAR function. For the object, CD99L2 was used in the present invention. CD99L2 (CD99 antigen-like 2) is a cell membrane protein belonging to the CD99 family, and CD99 family proteins are known to be mainly expressed in leukocyte, endothelial cells, etc. Functionally, these proteins have been reported to promote cell adhesion, cell migration, and the like (Pasello M, et al., J Cell Commun Signal. 2018; 12 (1): 55-68). In particular, CD99L2 has been reported to be involved in the extravasation of neutrophils, monocyte, and T cells in inflammatory conditions. In addition, it has been suggested that CD99L2 expressed in vascular endothelial cells may be involved in the extravasation of leukocyte (Seelige R, et al., J Immunol. 2013; 190 (3): 892-6). CD99L2 forms a heterodimer with CD99 (Nam G, et al., J Immunol. 2013; 191 (11): 5730-42), and CD99 protein has been reported to be involved in T cell co-stimulation. Therefore, there is also the possibility that CD99L2 may contribute to T cell activation (Oh KI, et al., Exp Mol Med. 2007; 39 (2): 176-84).

As a result, by designing and producing a CAR protein with a CD99L2 site, the present invention aims to present a new concept of CAR-T cells with enhanced functionality through T cell activation.

Accordingly, in one aspect, the present invention is directed to a chimeric antigen receptor (CAR) comprising
(a) an antigen binding domain;
(b) a backbone comprising an extracellular spacer domain and a transmembrane domain; and
(c) an intracellular signaling domain,

wherein the extracellular spacer domain comprises a CD99L2-derived extracellular domain, and wherein the transmembrane domain comprises a CD99L2-derived transmembrane domain.

As used herein, the term “backbone” refers to a region comprising an extracellular spacer domain and a transmembrane domain.

As used herein, the term “extracellular spacer domain” refers to a region connecting the antigen-binding domain to the transmembrane domain.

In the present invention, the extracellular spacer domain may comprise all or part of a CD99L2-derived extracellular domain, preferably a human CD99L2-derived extracellular domain. The CD99L2-derived extracellular domain may comprise all or part of the amino acid sequence represented by SEQ ID NO: 10, but is not limited thereto.

In the present invention, the transmembrane domain (TM) may comprise all or part of a CD99L2-derived transmembrane domain, preferably a human CD99L2-derived transmembrane domain. The CD99L2-derived transmembrane domain may comprise all or part of the amino acid sequence represented by SEQ ID NO: 11, but is not limited thereto.

In addition, in the present invention, the chimeric antigen receptor may further comprise a CD99-derived intracellular domain.

The CD99L2-derived intracellular domain may comprise all or part of the CD99L2-derived intracellular domain, and preferably comprises the amino acid sequence represented by SEQ ID NO: 12, but is not limited thereto.

In the present invention, the extracellular spacer domain may further comprise a hinge domain.

The hinge domain may be comprised of any oligopeptide or polypeptide, and may comprise 1 to 100 amino acid residues, and preferably 10 to 70 amino acid residues, but is not limited thereto.

In the present invention, the intracellular signaling domain is a portion located in the cytoplasm, which is the inside of the cell membrane of an immune cell, and is a region that activates the immune response of immune cells by transmitting a signal into the cells when the antigen-binding domain included in the extracellular domain binds to a target antigen.

In the present invention, the intracellular signaling domain is preferably at least one intracellular signaling domain selected from the group consisting of CD3 zeta (ζ), CD3 gamma (γ), CD3 delta (δ), CD3 epsilon (ε), FcR gamma, FcR beta, CD5, CD22, CD79a, CD79b, and CD66d, but is not limited thereto, and is more preferably CD3 zeta (ζ). The CD3 zeta (ζ) intracellular signaling domain according to the present invention may comprise the amino acid sequence of SEQ ID NO: 13 or the amino acid sequence of SEQ ID NO: 14 in which, glutamine (Q) which is the 14^{th} amino acid residue in the sequence of SEQ ID NO: 13, is substituted with lysine (K), but is not limited thereto.

In addition, the intracellular signaling domain according to the present invention may further comprise a co-stimulatory domain, but is not limited thereto. The co-stimulatory domain according to the present invention is preferably at least one co-stimulatory domain selected from the group consisting of CD2, CD7, CD27, CD28, CD30, CD40, 4-1BB (CD137), OX40 (CD134), ICOS, LFA-1, GITR, MyD88, DAP1, PD-1, LIGHT, NKG2C, B7-H3, and CD83 ligands, but is not limited thereto.

Preferably, the intracellular signaling domain according to the present invention comprises a CD3 zeta (Z) intracellular signaling domain comprising the amino acid sequence represented by SEQ ID NO: 13 or 14, and a 4-1BB co-stimulatory domain comprising the amino acid sequence represented by SEQ ID NO: 15, but is not limited thereto.

In particular, the chimeric antigen receptor according to the present invention may comprise at least one intracellular signaling domain and at least one co-stimulatory domain.

When the chimeric antigen receptor according to the present invention comprises at least one intracellular signaling domain and at least one co-stimulatory domain, at least one intracellular signaling domain and at least one co-stimulatory domain may be connected in series to each other. As such, each domain may be directly linked, or may be linked optionally or via an oligopeptide linker composed of 2 to 10 amino acid residues or a polypeptide linker, and the linker sequence preferably comprises a contiguous glycine-serine sequence.

In the present invention, the chimeric antigen receptor may further comprise a T-cell-immune-function-promoting factor, and examples of the T-cell-immune-function-promoting factor may comprise, but are not limited to, IL-7 (interleukin 7), IL-12, IL-15, IL-18, IL-21, and CCL19. Reference may be made to WO 2016/056228 A regarding the T-cell-immune-function-promoting factor.

In the present invention, the chimeric antigen receptor may further comprise an interleukin receptor chain comprising a JAK binding motif and a STAT 3/5 association motif, and an example thereof may include, but is not limited to, IL-2RB. In this regard, reference may be made to WO 2016/127257 A.

The first-generation CAR comprises an extracellular domain comprising a region that recognizes an antigen specifically expressed in cancer cells, a transmembrane domain, and an intracellular signaling domain, and uses only CD37 as the signaling domain. However, its therapeutic effect on cancer is insignificant, and the duration of the effect is short, which is undesirable. This first-generation CAR is specifically described in U.S. Pat. No. 6,319,494, which is incorporated herein by reference.

The second-generation CAR comprising a co-stimulatory domain (CD28 or CD137/4-1BB) and CD3ζ, which are coupled to each other, was prepared in order to improve the response to immune cells, and the number of CAR-containing immune cells remaining in the body was significantly increased compared to the first-generation CAR. The second-generation CAR used one co-stimulatory domain, whereas the third-generation CAR used two or more co-stimulatory domains. The co-stimulatory domain may be coupled with 4-1BB, CD28, OX40, etc. in order to achieve expansion and persistence of immune cells comprising CAR in vivo. The second-generation CAR is specifically described in U.S. Pat. Nos. 7,741,465, 7,446,190 and 9,212,229, and the third-generation CAR is specifically described in U.S. Pat. No. 8,822,647, all of which are incorporated herein by reference.

In the fourth-generation CAR, an additional gene encoding cytokine such as IL-12 or IL-15 is included to allow additional expression of the CAR-based immune protein of cytokine, and the fifth-generation CAR further includes an interleukin receptor chain such as IL-2RB in order to enhance immune cells. The fourth-generation CAR is specifically described in U.S. Pat. No. 10,316,102, and the fifth-generation CAR is specifically described in U.S. Pat. No. 10,336,810, both of which are incorporated herein by reference.

In the present invention, the antigen-binding domain may comprise, but is not limited to, an antibody or antigen-binding fragment thereof that specifically binds to an antigen selected from the group consisting of:
4-1BB, B cell maturation antigen (BCMA), B-cell activating factor (BAFF), B7-H3, B7-H6, carbonic anhydrase 9 (CA9; also known as CAIX or G250), cancer/testis antigen 1B (CTAG1B; also known as NY-ESO-1 or LAGE2B), carcinoembryonic antigen (CEA), cyclin, cyclin A2, cyclin B1, C-C motif chemokine ligand 1 (CCL-I), CCR4, CD3, CD4, CD19, CD20, CD22, CD23, CD24, CD30, CD33, CD38, CD40, CD44, CD44v6, CD44v7/8, CD52, CD58, CD62, CD79A, CD79B, CD80, CD123, CD133, CD138, CD171, chondroitin sulfate proteoglycan 4 (CSPG4), claudin-18 (CLDN18), CLDN6, cytotoxic T-lymphocyte-associated protein 4 (CTLA-4), tyrosine-protein kinase Met (c-Met), DLL3, epidermal growth factor receptor (EGFR), truncated epidermal growth factor receptor (tEGFR), type III epidermal growth factor receptor mutation (EGFRvlll), epithelial glycoprotein 2 (EPG-2), epithelial glycoprotein 40 (EPG-40), ephrin B2, ephrin receptor A2 (EPHA2), estrogen receptor, Fc receptor, Fc-receptor-like 5 (FCRL5; also known as Fc receptor homolog 5 or FCRH5), fibroblast growth factor 23 (FGF23), folate binding protein (FBP), folate receptor alpha (FOLR1), folate receptor beta (FOLR2), GD2 (ganglioside GD2, O-acetylated GD2 (OGD2)), ganglioside GD3, glycoprotein 100 (gp100), glypican-3 (GPC3), G protein-coupled receptor 5D (GPCR5D), granulocyte-macrophage colony-stimulating factor (GM-CSF), Her2/neu (receptor tyrosine kinase erb-B2), Her3 (erb-B3), Her4 (erb-B4), erbB dimers, human high-molecular-weight melanoma-associated antigen (HMW-MAA), hepatitis B surface antigen (HBsAg), human leukocyte antigen A1 (HLA-A1), human leukocyte antigen A2 (HLA-A2), IL-22 receptor alpha (IL-22Ra), IL-13 receptor alpha 2 (IL-13Ra2), inducible T-cell costimulator (ICOS), insulin-like growth factor 1 receptor (IGF-1 receptor), integrin avß6, interferon receptor, IFNγ receptor (IFNγR), interleukin-2 receptor (IL-2R), interleukin-4 receptor (IL-4R), interleukin-5 receptor (IL-5R), interleukin-6 receptor (IL-6R), interleukin-17 receptor A (IL-17RA), interleukin-31 receptor (IL-31R), interleukin-36 receptor (IL-36R), kinase insert domain receptor (kdr), L1 cell adhesion molecule (L1-CAM), CE7 epitope of L1-CAM, leucine-rich repeat-containing 8 family member A (LRRC8A), Lewis Y, lymphocyte-activation gene 3 (LAG3), melanoma-associated antigen (MAGE) A1, MAGEA3, MAGEA6, MAGEA10, mesothelin (MSLN), murine cytomegalovirus (CMV), mucin 1 (MUC1), natural killer group 2 member D (NKG2D) ligands, melan A (MART-I), nerve growth factor (NGF), neural cell adhesion molecule (NCAM), neuropilin-1 (NRP-1), neuropilin-2 (NRP-2), oncofetal antigen, PD-L1, preferentially expressed antigen of melanoma (PRAME), progesterone receptor, prostate-specific antigen, prostate stem cell antigen (PSCA), prostate-specific membrane antigen (PSMA), receptor activator of nuclear factor kappa-B ligand (RANKL), receptor-tyrosine-kinase-like orphan receptor 1 (ROR1), SLAM family member 7 (SLAMF7), survivin, trophoblast glycoprotein (TPBG; also known as 5T4), tumor-associated glycoprotein 72 (TAG72), tyrosine-related protein 1 (TRP1; also known as TYRP1 or gp75), tyrosine-related protein 2 (TRP2; also known as dopachrome tautomerase, dopachrome delta-isomerase or DCT), and Wilms' tumor 1 (WT1).

In the present invention, the “fragment” of an antibody is a fragment having an antigen-binding function, and is used to have a meaning comprising scFv, Fab, F(ab′)2, Fv, and nanobody fragments.

A “single-chain Fv” or “scFv (single chain variable fragment)” antibody fragment comprises the VH and VL domains of an antibody, and such domains are present within a single polypeptide chain. The Fv polypeptide may further comprise a polypeptide linker between the VH and VL domains that enables scFv to form the desired structure for antigen binding.

An “Fv” fragment is an antibody fragment comprising complete antibody recognition and binding sites. This region is comprised of a dimer in which one heavy-chain variable domain and one light-chain variable domain are tightly and substantially covalently associated with, for example, an scFv.

A “Fab” fragment comprises the variable and constant domains of a light chain and the variable and first constant domains (CH1) of a heavy chain. “F(ab′)2” antibody fragments generally comprise a pair of Fab fragments that are covalently linked near the carboxy terminus thereof by a hinge cysteine therebetween.

A “nanobody” is a fragment comprising a monomeric variable antibody domain. It is mainly comprised of a low-molecular-weight fragment derived from a camelid antibody domain that shows target specificity only with a monomeric heavy chain.

In the present invention, the antigen-binding fragment is a single-chain variable fragment (scFv) or nanobody of an antibody.

In the present invention, the antigen-binding domain preferably comprises an anti-CD19 antibody or an scFv thereof, and the scFv of the anti-CD19 antibody comprises the amino acid sequence represented by SEQ ID NO: 8, but is not limited thereto.

In the present invention, the chimeric antigen receptor further comprises a signal peptide (SP) at the N-terminus of the antigen-binding domain. In the present invention, the signal peptide may be derived from a molecule selected from the group consisting of CD8a, GM-CSF receptor a, Ig-kappa, and IgG1 heavy chain, but is not limited thereto, and is preferably a CD8a signal peptide, and the CD8a signal peptide may comprise the amino acid sequence represented by SEQ ID NO: 7.

In a preferred embodiment, the chimeric antigen receptor according to the present invention comprises
a CD99L2-derived extracellular domain represented by SEQ ID NO: 10;
a CD99L2-derived transmembrane domain represented by SEQ ID NO: 11; and
a CD99L2-derived intracellular domain represented by SEQ ID NO: 12.

In addition, the chimeric antigen receptor according to the present invention may further comprises a 4-1BB co-stimulatory domain represented by SEQ ID NO: 15;
a CD3 zeta (Z) intracellular signaling domain represented by SEQ ID NO: 13 or 14; and/or
a CD8 signal peptide represented by SEQ ID NO: 7, but is not limited thereto.

In an exemplary embodiment of the present invention, the chimeric antigen receptor comprising an antigen-binding domain for CD19 may comprise the amino acid sequence represented by SEQ ID NO: 2 or 3, or a variant thereof having sequence identity of 80% or more, preferably 90% or more, more preferably 95% or more, and most preferably 99% or more to the amino acid sequence described above.

In another aspect, the present invention is directed to a nucleic acid encoding the chimeric antigen receptor.

As used herein, the term “nucleic acid” is intended to encompass DNA (gDNA and cDNA) and RNA molecules, and the nucleotide, which is the basic building block in a nucleic acid, includes not only naturally occurring nucleotides but also analogs in which sugar or base sites are modified. The sequences of the nucleic acids encoding the chimeric antigen receptors of the present invention or their respective domains can be modified. Said modifications include additions, deletions, or non-conservative or conservative substitutions of nucleotides.

The nucleic acid (polynucleotide) encoding the chimeric antigen receptor according to the present invention may be modified through codon optimization, which is due to the degeneracy of codons, and the presence of many nucleotide sequences encoding the polypeptides or variant fragments thereof may be well understood by those of ordinary skill in the art. Some of these polynucleotides (nucleic acids) retain minimal homology with the nucleotide sequence of any naturally occurring gene. In particular, polynucleotides that vary due to differences in codon usage, for example, polynucleotides optimized for codon selection in humans, primates and/or mammals, are preferred.

In the present invention, the nucleic acid encoding the chimeric antigen receptor comprises
a nucleotide sequence encoding the CD99L2-derived extracellular domain and represented by SEQ ID NO: 19; and
a nucleotide sequence encoding the CD99L2-derived transmembrane domain and represented by SEQ ID NO: 20; and further comprises
a nucleotide sequence encoding the CD99L2-derived intracellular domain and represented by SEQ ID NO: 21;
a nucleotide sequence encoding the 4-1BB co-stimulatory domain and represented by SEQ ID NO: 25 or 26;
a nucleotide sequence encoding the CD3 zeta (2) intracellular signaling domain and represented by SEQ ID NO: 22, 23 or 24; and/or
a nucleotide sequence encoding the CD8 signal peptide and represented by SEQ ID NO: 16, but the present invention is not limited thereto.

Preferably, the nucleic acid further comprises a nucleotide sequence encoding the single-chain variable fragment (scFv) of an anti-CD19 antibody and represented by SEQ ID NO: 17.

In one embodiment of the present invention, the nucleic acid sequence encoding the chimeric antigen receptor can comprise the nucleotide sequence represented by SEQ ID NO: 5 or 6, or a variant thereof having sequence identity of 80% or more, preferably 90% or more, more preferably 95% or more, and most preferably 99% or more to the nucleotide sequence described above.

In still another aspect, the present invention is directed to an expression vector comprising the nucleic acid and a virus comprising the expression vector.

As used herein, the term “vector” refers to a nucleic acid molecule capable of transferring or transporting another nucleic acid molecule. The transferred nucleic acid is generally linked to a vector nucleic acid molecule, and, for example is inserted into a vector nucleic acid molecule. The vector may comprise a sequence that directs autonomous replication in the cells, or may comprise a sequence sufficient to permit integration into host cell DNA. The vector may be selected from the group consisting of DNA, RNA, plasmids, lentiviral vectors, adenoviral vectors, and retroviral vectors, but is not limited thereto.

In the present invention, the nucleic acid or the vector is transfected into a viral packaging cell line. A variety of different techniques that are commonly used to introduce exogenous nucleic acid (DNA or RNA) into prokaryotic or eukaryotic host cells for “transfection”, for example, electroporation, calcium phosphate precipitation, DEAE-dextran transfection, lipofection, etc., may be used.

In the present invention, the virus produced from the viral packaging cell line is transduced into immune cells. The nucleic acid of the virus that is “transduced” into the cells is used to produce a chimeric antigen receptor protein, either in the state of being inserted into the genome of the cells or not.

In yet another aspect, the present invention is directed to an immune cell expressing the chimeric antigen receptor on the surface thereof.

In the present invention, the immune cells may be T cells, NK cells, NKT cells, or macrophages, but are not limited thereto, and are preferably T cells.

The immune cells expressing the chimeric antigen receptor according to the present invention may be CAR-T cells (chimeric antigen receptor T cells), CAR-NK cells (chimeric antigen receptor natural killer cells), CAR-NKT cells (chimeric antigen receptor natural killer T cells), or CAR-macrophages (chimeric antigen receptor macrophages).

In the present invention, the T cells may be selected from the group consisting of CD4-positive T cells, CD8-positive cytotoxic T lymphocytes (CTL), gamma-delta T cells, tumor-infiltrating lymphocytes (TIL), and T cells isolated from peripheral blood mononuclear cells (PBMCs).

In still yet another aspect, the present invention is directed to a composition for treating cancer comprising the immune cells (e.g. T cells) expressing the chimeric antigen receptor.

In the present invention, “cancer” and “tumor” are used to have the same meaning, and refer to or mean a physiological condition in mammals, typically characterized by unregulated cell growth and proliferation.

The types of cancer that may be treated using the CAR of the present invention include not only vascularized tumors but also non-vascularized or not yet vascularized tumors. The cancer may include non-solid tumors (e.g. hematologic tumors such as leukemia and lymphoma), or may include solid tumors. The types of cancer that may be treated using the CAR of the present invention include carcinoma, blastoma, sarcoma, and certain leukemia or lymphoid malignancies, benign and malignant tumors, for example, sarcoma, carcinoma and melanoma, but are not limited thereto. Also included are adult tumors/cancer and pediatric tumors/cancer.

Hematologic cancer is cancer of the blood or bone marrow. Examples of hematologic (or hematopoietic) cancer include acute leukemia (e.g. acute lymphocytic leukemia, acute myeloid leukemia, myeloblastic leukemia, prolymphocytic leukemia, myeloid monocytic leukemia, monocytic leukemia, and erythroleukemia), chronic leukemia (e.g. chronic lymphocytic (granulocytic) leukemia, chronic myeloid leukemia, and chronic lymphocytic leukemia), polycythemia vera, lymphoma, Hodgkin's disease, non-Hodgkin's lymphoma (delayed and high-stage forms), multiple myeloma, Waldenstrom's macroglobulinemia, heavy-chain disease, myelodysplastic syndrome, hair-cell leukemia, and leukemia including myelodysplasia.

Solid tumors are abnormal masses of tissue that generally do not include cysts or liquid zones. Solid tumors may be benign or malignant. Different types of solid tumors are named for the types of cells that form them (e.g. sarcomas, carcinomas, and lymphomas). Examples of solid tumors such as sarcomas and carcinomas include fibrosarcoma, myxosarcoma, liposarcoma, chondrosarcoma, osteosarcoma, other sarcomas, synovioma, mesothelioma, Ewing tumor, leiomyosarcoma, rhabdomyosarcoma, rectal carcinoma, lymphoid malignancy, colorectal cancer, stomach cancer, pancreatic cancer, breast cancer, lung cancer, ovarian cancer, prostate cancer, pharyngeal cancer, hepatocellular carcinoma, squamous cell carcinoma, basal cell carcinoma, adenocarcinoma, sweat gland carcinoma, medullary thyroid carcinoma, papillary thyroid carcinoma, pheochromocytoma, sebaceous adenocarcinoma, papillary carcinoma, papillary adenocarcinoma, medullary carcinoma, bronchial carcinoma, renal cell carcinoma, liver tumor, cholangiocarcinoma, choriocarcinoma, Wilms' tumor, cervical cancer, testicular tumors, seminoma, bladder cancer, melanoma, and CNS tumors (e.g. gliomas (e.g. brainstem glioma and mixed glioma), glioblastoma (also known as glioblastoma multiforme), astrocytoma, CNS lymphoma, germinoma, medullary blastoma, schwannoma craniopharyngioma, ependymoma, pinealoma, hemangioblastoma, acoustic neuroma, oligodendroglioma, meningioma, neuroblastoma, retinoblastoma, and brain metastasis).

The therapeutic composition of the present invention is a composition for the prevention or treatment of cancer, and the term “prevention” of the present invention refers to any action that inhibits cancer or delays the progression of cancer by administration of the composition of the present invention, and “treatment” means inhibiting the development of cancer and alleviating or eliminating symptoms thereof. The pharmaceutical composition comprising the immune cells expressing the chimeric antigen receptor according to the present invention may further comprise a pharmaceutically acceptable excipient. Examples of such excipients include surfactants, preferably nonionic surfactants such as polysorbate series, buffers such as neutral buffered saline, phosphate buffered saline and the like, sugars or sugar alcohols such as glucose, mannose, sucrose, dextran, mannitol and the like, amino acids, proteins or polypeptides such as glycine, histidine and the like, antioxidants, chelating agents such as EDTA or glutathione, penetrants, supplements, and preservatives, but are not limited thereto.

The composition of the present invention may be formulated using methods known in the art in order to provide rapid, sustained or delayed release of the active ingredient after administration to a mammal other than a human. A formulation may be in the form of a powder, granule, tablet, emulsion, syrup, aerosol, soft or hard gelatin capsule, sterile injectable solution, or sterile powder.

In further another aspect, the present invention is directed to a method of treating cancer comprising administering immune cells expressing the chimeric antigen receptor to a subject.

The present invention is also directed to the use of the immune cells for the treatment of cancer.

The present invention is also directed to the use of the immune cells for the manufacture of a medicament for the treatment of cancer.

The subject may be a mammal having a tumor, particularly a human, but is not limited thereto.

The immune cells expressing the chimeric antigen receptor according to the present invention or the composition comprising the same may be administered orally or through infusion, intravenous injection, intramuscular injection, subcutaneous injection, intraperitoneal injection, intrarectal administration, topical administration, intranasal injection, etc., but the present invention is not limited thereto.

The dosage of the active ingredient may be appropriately selected depending on various factors, such as the route of administration, the age, gender, and weight of the patient, and the severity of the disease, and the therapeutic composition according to the present invention may be administered in combination with a known compound effective at preventing, ameliorating or treating cancer symptoms.

Hereinafter, the present invention will be described in more detail with reference to examples. However, it will be obvious to those skilled in the art that these examples are provided only for illustration of the present invention, and should not be construed as limiting the scope of the present invention.

### EXAMPLE 1: Materials and Methods

### EXAMPLE 1-1 Mouse and Cell Line

Immunodeficiency NSG mice were purchased from the Jackson laboratory. Raji lymphoma cells were purchased from ATCC.

### EXAMPLE 1-2. Fabrication of Lentiviral Vector for CAR Expression

CD19-targeted CD8 backbone CAR (h19BBz) ORF cDNA was commissioned for DNA synthesis according to previously published sequences (U.S. Patent US 2013/0287748 A1) (Integrated DNA Technologies). CD19 targeting CD99L2 backbone CAR ORF cDNAS (FL2LBBz, FL2PBBz) were generated from the human CD99L2 ORF sequence (NM_031462. 4) from the NCBI database. The sequences of some extracellular, transmembrane and intracellular parts of CD99L2 were extracted and ligated with the sequences of human 41BB intracellular part, human CD3 zeta chain intracellular part via codon optimization and DNA synthesis (Integrated DNA Technologies), and then ligated with anti-CD19 scFv (clone FMC63) by PCR. The lentiviral vector for CAR expression was a modification of the pCDH-EF1 (Addgene #72266) vector, and was constructed by cloning each CAR ORF cDNA into BamHI/Sall restriction enzyme sites. The amino acid sequences and nucleotide sequences of each CAR protein are listed in Table 1 and Table 2 below.

TABLE 1
Amino acid sequences of CAR protein

| | | SEQ ID |
|---|---|---|
| Classification | Sequence | NO: |
| h19BBz | MALPVTALLL PLALLLHAAR PDIQMTQTTS SLSASLGDRV TISCRASQDI | 1 |
| | SKYLNWYQQK PDGTVKLLIY HTSRLHSGVP SRFSGSGSGT DYSLTISNLE | |
| | QEDIATYFCQ QGNTLPYTFG GGTKLEITGG GGSGGGGSGG GGSEVKLQES | |
| | GPGLVAPSQS LSVTCTVSGV SLPDYGVSWI RQPPRKGLEW LGVIWGSETT | |
| | YYNSALKSRL TIIKDNSKSQ VFLKMNSLQT DDTAIYYCAK HYYYGGSYAM | |
| | DYWGQGTSVT VSSTTTPAPR PPTPAPTIAS QPLSLRPEAC RPAAGGAVHT | |
| | RGLDFACDIY IWAPLAGTCG VLLLSLVITL YCKRGRKKLL YIFKQPFMRP | |
| | VQTTQEEDGC SCRFPEEEEG GCELRVKFSR SADAPAYKQG QNQLYNELNL | |
| | GRREEYDVLD KRRGRDPEMG GKPRRKNPQE GLYNELQKDK MAEAYSEIGM | |
| | KGERRRGKGH DGLYQGLSTA TKDTYDALHM QALPPR | |
| FL2PBBz | MALPVTALLL PLALLLHAAR PDIQMTQTTS SLSASLGDRV TISCRASQDI | 2 |
| | SKYLNWYQQK PDGTVKLLIY HTSRLHSGVP SRFSGSGSGT DYSLTISNLE | |
| | QEDIATYFCQ QGNTLPYTFG GGTKLEITGG GGSGGGGSGG GGSEVKLQES | |
| | GPGLVAPSQS LSVTCTVSGV SLPDYGVSWI RQPPRKGLEW LGVIWGSETT | |
| | YYNSALKSRL TIIKDNSKSQ VFLKMNSLQT DDTAIYYCAK HYYYGGSYAM | |
| | DYWGQGTSVT VSSGGFSDKD LEDIVGGGEY KPDKGKGDGR YGSNDDPGSG | |
| | MVAEPGTIAG VASALAMALI GAVSSYISYQ QKKFCFSIQK RGRKKLLYIF | |
| | KQPFMRPVQT TQEEDGCSCR FPEEEEGGCE LRVKFSRSAD APAYQQGQNQ | |
| | LYNELNLGRR EEYDVLDKRR GRDPEMGGKP RRKNPQEGLY NELQKDKMAE | |
| | AYSEIGMKGE RRRGKGHDGL YQGLSTATKD TYDALHMQAL PPR | |
| FL2LBBz | MALPVTALLL PLALLLHAAR PDIQMTQTTS SLSASLGDRV TISCRASQDI | 3 |
| | SKYLNWYQQK PDGTVKLLIY HTSRLHSGVP SRFSGSGSGT DYSLTISNLE | |
| | QEDIATYFCQ QGNTLPYTFG GGTKLEITGG GGSGGGGSGG GGSEVKLQES | |
| | GPGLVAPSQS LSVTCTVSGV SLPDYGVSWI RQPPRKGLEW LGVIWGSETT | |
| | YYNSALKSRL TIIKDNSKSQ VFLKMNSLQT DDTAIYYCAK HYYYGGSYAM | |
| | DYWGQGTSVT VSSGGFSDKD LEDIVGGGEY KPDKGKGDGR YGSNDDPGSG | |
| | MVAEPGTIAG VASALAMALI GAVSSYISYQ QKKFCFSIQQ GLNADYVKGE | |
| | NLEAVVCEEP QVKYSTLHTQ SAEPPPPPEP ARIKRGRKKL LYIFKQPFMR | |
| | PVQTTQEEDG CSCRFPEEEE GGCELRVKFS RSADAPAYQQ GQNQLYNELN | |
| | LGRREEYDVL DKRRGRDPEM GGKPRRKNPQ EGLYNELQKD KMAEAYSEIG | |
| | MKGERRRGKG HDGLYQGLST ATKDTYDALH MQALPPR | |

TABLE 2
Nucleotide sequences of CAR protein

| | | SEQ ID |
|---|---|---|
| Classification | Sequence | NO: |
| h19BBz | atggccttaccagtgaccgccttgctcctgccgctggccttgctgctccacgcc | 4 |
| | gccaggccggacatccagatgacacagactacatcctccctgtctgcctctctg | |
| | ggagacagagtcaccatcagttgcagggcaagtcaggacattagtaaatattta | |
| | aattggtatcagcagaaaccagatggaactgttaaactcctgatctaccataca | |
| | tcaagattacactcaggagtcccatcaaggttcagtggcagtgggtctggaaca | |
| | gattattctctcaccattagcaacctggagcaagaagatattgccacttacttt | |
| | tgccaacagggtaatacgcttccgtacacgttcggaggggggaccaagctggag | |
| | atcacaggtggcggtggctcgggcggtggtgggtcgggtggcggcggatctgag | |
| | gtgaaactgcaggagtcaggacctggcctggtggcgccctcacagagcctgtcc | |
| | gtcacatgcactgtctcaggggtctcattacccgactatggtgtaagctggatt | |
| | cgccagcctccacgaaagggtctggagtggctgggagtaatatggggtagtgaa | |
| | accacatactataattcagctctcaaatccagactgaccatcatcaaggacaac | |
| | tccaagagccaagttttcttaaaaatgaacagtctgcaaactgatgacacagcc | |
| | atttactactgtgccaaacattattactacggtggtagctatgctatggactac | |
| | tggggccaaggaacctcagtcaccgtctcctcaaccacgacgccagcgccgcga | |
| | ccaccaacaccggcgcccaccatcgcgtcgcagcccctgtccctgcgcccagag | |
| | gcgtgccggccagcggcggggggcgcagtgcacacgagggggctggacttcgcc | |
| | tgtgatatctacatctgggcgcccttggccgggacttgtggggtccttctcctg | |
| | tcactggttatcaccctttactgcaaacggggcagaaagaaactcctgtatata | |
| | ttcaaacaaccatttatgagaccagtacaaactactcaagaggaagatggctgt | |
| | agctgccgatttccagaagaagaagaaggaggatgtgaactgagagtgaagttc | |
| | agcaggagcgcagacgcccccgcgtacaagcagggccagaaccagctctataac | |
| | gagctcaatctaggacgaagagaggagtacgatgttttggacaagagacgtggc | |
| | cgggaccctgagatggggggaaagccgagaaggaagaaccctcaggaaggcctg | |
| | tacaatgaactgcagaaagataagatggcggaggcctacagtgagattgggatg | |
| | aaaggcgagcgccggaggggcaaggggcacgatggcctttaccagggtctcagt | |
| | acagccaccaaggacacctacgacgcccttcacatgcaggccctgccccctcgc | |
| | taa | |
| FL2PBBz | atggccttaccagtgaccgccttgctcctgccgctggccttgctgctccacgcc | 5 |
| | gccaggccggacatccagatgacacagactacatcctccctgtctgcctctctg | |
| | ggagacagagtcaccatcagttgcagggcaagtcaggacattagtaaatattta | |
| | aattggtatcagcagaaaccagatggaactgttaaactcctgatctaccataca | |
| | tcaagattacactcaggagtcccatcaaggttcagtggcagtgggtctggaaca | |
| | gattattctctcaccattagcaacctggagcaagaagatattgccacttacttt | |
| | tgccaacagggtaatacgcttccgtacacgttcggaggggggaccaagctggag | |
| | atcacaggtggcggtggctcgggcggtggtgggtcgggtggcggcggatctgag | |
| | gtgaaactgcaggagtcaggacctggcctggtggcgccctcacagagcctgtcc | |
| | gtcacatgcactgtctcaggggtctcattacccgactatggtgtaagctggatt | |
| | cgccagcctccacgaaagggtctggagtggctgggagtaatatggggtagtgaa | |
| | accacatactataattcagctctcaaatccagactgaccatcatcaaggacaac | |
| | tccaagagccaagttttcttaaaaatgaacagtctgcaaactgatgacacagcc | |
| | atttactactgtgccaaacattattactacggtggtagctatgctatggactac | |
| | tggggccaaggaacctcagtcaccgtctcctcaggcggcttcagcgacaaggac | |
| | ctggaagatatcgttggcggcggagagtacaagcccgacaaaggcaaaggcgac | |
| | ggcagatacggcagcaacgatgatcctggctctggcatggtggccgagcctgga | |
| | acaattgctggcgtggcatctgccctggccatggctcttattggagccgtgtcc | |
| | agctacatcagctaccagcagaagaagttctgcttcagcatccagaagcggggc | |
| | agaaagaagctgctgtacatcttcaagcagcccttcatgcggcccgtgcagacc | |
| | acacaagaggaagatggctgctcctgcagattccccgaggaagaagaaggcggc | |
| | tgcgagctgagagtgaagttcagcagatccgccgacgctcccgcttatcagcag | |
| | ggacagaaccagctgtacaacgagctgaacctggggagaagagaagagtacgac | |
| | gtgctggacaagcggagaggcagagatcctgagatgggcggcaagcccagacgg | |
| | aagaatcctcaagagggcctgtataatgagctgcagaaagacaagatggccgag | |
| | gcctacagcgagatcggaatgaagggcgagcgcagaagaggcaagggacacgat | |
| | ggactgtaccagggcctgagcaccgccaccaaggatacctatgatgccctgcac | |
| | atgcaggccctgcctccaagataa | |
| FL2LBBz | atggccttaccagtgaccgccttgctcctgccgctggccttgctgctccacgcc | 6 |
| | gccaggccggacatccagatgacacagactacatcctccctgtctgcctctctg | |
| | ggagacagagtcaccatcagttgcagggcaagtcaggacattagtaaatattta | |
| | aattggtatcagcagaaaccagatggaactgttaaactcctgatctaccataca | |
| | tcaagattacactcaggagtcccatcaaggttcagtggcagtgggtctggaaca | |
| | gattattctctcaccattagcaacctggagcaagaagatattgccacttacttt | |
| | tgccaacagggtaatacgcttccgtacacgttcggaggggggaccaagctggag | |
| | atcacaggtggcggtggctcgggcggtggtgggtcgggtggcggcggatctgag | |
| | gtgaaactgcaggagtcaggacctggcctggtggcgccctcacagagcctgtcc | |
| | gtcacatgcactgtctcaggggtctcattacccgactatggtgtaagctggatt | |
| | cgccagcctccacgaaagggtctggagtggctgggagtaatatggggtagtgaa | |
| | accacatactataattcagctctcaaatccagactgaccatcatcaaggacaac | |
| | tccaagagccaagttttcttaaaaatgaacagtctgcaaactgatgacacagcc | |
| | atttactactgtgccaaacattattactacggtggtagctatgctatggactac | |
| | tggggccaaggaacctcagtcaccgtctcctcaggcggcttcagcgacaaggac | |
| | ctggaagatatcgttggcggcggagagtacaagcccgacaaaggcaaaggcgac | |
| | ggcagatacggcagcaacgatgatcctggctctggcatggtggccgagcctgga | |
| | acaattgctggcgtggcatctgccctggccatggctcttattggagccgtgtcc | |
| | agctacatcagctaccagcagaagaagttctgcttcagcatccagcagggcctg | |
| | aacgccgattacgtgaagggcgagaatctggaagccgtcgtgtgcgaggaaccc | |
| | caagtgaagtacagcaccctgcacacccagtctgccgaacctccacctcctcca | |
| | gaacctgccagaatcaagcggggcagaaagaagctgctgtacatcttcaagcag | |
| | cccttcatgcggcccgtgcagaccacacaagaggaagatggctgctcctgcaga | |
| | ttccccgaggaagaagaaggcggctgcgagctgagagtgaagttctccagatcc | |
| | gccgacgctcccgcttatcagcagggacagaaccagctgtacaacgagctgaac | |
| | ctggggagaagagaagagtacgacgtgctggacaagcggagaggcagagatcct | |
| | gagatgggcggcaagcccagacggaagaatcctcaagagggcctgtataatgag | |
| | ctgcagaaagacaagatggccgaggcctacagcgagatcggcatgaagggcgaa | |
| | cgcagaagaggcaagggacacgatggcctgtatcagggcctgtctaccgccacc | |
| | aaggacacctatgatgccctgcacatgcaggctctgcctccaagataa | |

The amino acid and nucleotide sequences of each domain constituting the CAR protein are as described in Tables 3 and 4 below.

TABLE 3
Amino acid sequences of each domain constituting CAR protein

| | | | SEQ |
|---|---|---|---|
| | | | ID |
| Classification | | Sequence | NO: |
| signal | hCD8L | MALPVTALLLPLALLLHAARP | 7 |
| peptide | | | |
| antigen- | αCD19 scFv | DIQMTQTTSSLSASLGDRVTISCRASQDISKYLNWYQQKPDGTV | 8 |
| binding | | KLLIYHTSRLHSGVPSRFSGSGSGTDYSLTISNLEQEDIATYFC | |
| domain | | QQGNTLPYTFGGGTKLEITGGGGSGGGGSGGGGSEVKLQESGPG | |
| | | LVAPSQSLSVTCTVSGVSLPDYGVSWIRQPPRKGLEWLGVIWGS | |
| | | ETTYYNSALKSRLTIIKDNSKSQVFLKMNSLQTDDTAIYYCAKH | |
| | | YYYGGSYAMDYWGQGTSVTVSS | |
| backbone | CD8 EC + TM | TTTPAPRPPTPAPTIASQPLSLRPEACRPAAGGAVHTRGLDFAC | 9 |
| | | DIYIWAPLAGTCGVLLLSLVITLYC | |
| | CD99L2 EC | GGFSDKDLEDIVGGGEYKPDKGKGDGRYGSNDDPGSGMVAEPG | 10 |
| | CD99L2 TM | TIAGVASALAMALIGAVSSYISYQQKKFCFSIQ | 11 |
| | CD99L2 | QGLNADYVKGENLEAVVCEEPQVKYSTLHTQSAEPPPPPEPARI | 12 |
| | intracellular | | |
| | domain | | |
| intracellular | CD3 zeta | RVKFSRSADAPAYQQGQNQLYNELNLGRREEYDVLDKRRGRDPE | 13 |
| signaling | intracellular | MGGKPRRKNPQEGLYNELQKDKMAEAYSEIGMKGERRRGKGHDG | |
| domain | signaling | LYQGLSTATKDTYDALHMQALPPR | |
| | domain | | |
| | (wild type) | | |
| | CD3 zeta | RVKFSRSADAPAYKQGQNQLYNELNLGRREEYDVLDKRRGRDPE | 14 |
| | intracellular | MGGKPRRKNPQEGLYNELQKDKMAEAYSEIGMKGERRRGKGHDG | |
| | signaling | LYQGLSTATKDTYDALHMQALPPR | |
| | domain | | |
| | (mutant) | | |
| | 4-1BB | KRGRKKLLYIFKQPFMRPVQTTQEEDGCSCRFPEEEEGGCEL | 15 |
| | CO- | | |
| | stimulatory | | |
| | domain | | |

TABLE 4
Nucleotide sequences of each domain constituting CAR protein

| | | | SEQ |
|---|---|---|---|
| | | | ID |
| Classification | | Sequence | NO: |
| signal | hCD8L | atggccttaccagtgaccgccttgctcctgccgctggccttgct | 16 |
| peptide | | gctccacgccgccaggccg | |
| antigen- | αCD19 scFv | gacatccagatgacacagactacatcctccctgtctgcctctct | 17 |
| binding | | gggagacagagtcaccatcagttgcagggcaagtcaggacatta | |
| domain | | gtaaatatttaaattggtatcagcagaaaccagatggaactgtt | |
| | | aaactcctgatctaccatacatcaagattacactcaggagtccc | |
| | | atcaaggttcagtggcagtgggtctggaacagattattctctca | |
| | | ccattagcaacctggagcaagaagatattgccacttacttttgc | |
| | | caacagggtaatacgcttccgtacacgttcggaggggggaccaa | |
| | | gctggagatcacaggtggcggtggctcgggcggtggtgggtcgg | |
| | | gtggcggcggatctgaggtgaaactgcaggagtcaggacctggc | |
| | | ctggtggcgccctcacagagcctgtccgtcacatgcactgtctc | |
| | | aggggtctcattacccgactatggtgtaagctggattcgccagc | |
| | | ctccacgaaagggtctggagtggctgggagtaatatggggtagt | |
| | | gaaaccacatactataattcagctctcaaatccagactgaccat | |
| | | catcaaggacaactccaagagccaagttttcttaaaaatgaaca | |
| | | gtctgcaaactgatgacacagccatttactactgtgccaaacat | |
| | | tattactacggtggtagctatgctatggactactggggccaagg | |
| | | aacctcagtcaccgtctcctca | |
| backbone | CD8 EC + TM | accacgacgccagcgccgcgaccaccaacaccggcgcccaccat | 18 |
| | | cgcgtcgcagcccctgtccctgcgcccagaggcgtgccggccag | |
| | | cggcggggggcgcagtgcacacgagggggctggacttcgcctgt | |
| | | gatatctacatctgggcgcccttggccgggacttgtggggtcct | |
| | | tctcctgtcactggttatcaccctttactgc | |
| | CD99L2 EC | ggcggcttcagcgacaaggacctggaagatatcgttggcggcgg | 19 |
| | | agagtacaagcccgacaaaggcaaaggcgacggcagatacggca | |
| | | gcaacgatgatcctggctctggcatggtggccgagcctgga | |
| | CD99L2 TM | acaattgctggcgtggcatctgccctggccatggctcttattgg | 20 |
| | | agccgtgtccagctacatcagctaccagcagaagaagttctgct | |
| | | tcagcatccag | |
| | CD99L2 | cagggcctgaacgccgattacgtgaagggcgagaatctggaagc | 21 |
| | intracellular | cgtcgtgtgcgaggaaccccaagtgaagtacagcaccctgcaca | |
| | signaling | cccagtctgccgaacctccacctcctccagaacctgccagaatc | |
| | domain | | |
| intra- | CD3 zeta | agagtgaagttctccagatccgccgacgctcccgcttatcagca | 22 |
| cellular | intracellular | gggacagaaccagctgtacaacgagctgaacctggggagaagag | |
| signaling | signaling | aagagtacgacgtgctggacaagcggagaggcagagatcctgag | |
| domain | domain | atgggcggcaagcccagacggaagaatcctcaagagggcctgta | |
| | (wild type) | taatgagctgcagaaagacaagatggccgaggcctacagcgaga | |
| | | tcggcatgaagggcgaacgcagaagaggcaagggacacgatggc | |
| | | ctgtatcagggcctgtctaccgccaccaaggacacctatgatgc | |
| | | cctgcacatgcaggctctgcctccaagataa | |
| | | agagtgaagttcagcagatccgccgacgctcccgcttatcagca | 23 |
| | | gggacagaaccagctgtacaacgagctgaacctggggagaagag | |
| | | aagagtacgacgtgctggacaagcggagaggcagagatcctgag | |
| | | atgggcggcaagcccagacggaagaatcctcaagagggcctgta | |
| | | taatgagctgcagaaagacaagatggccgaggcctacagcgaga | |
| | | tcggaatgaagggcgagcgcagaagaggcaagggacacgatgga | |
| | | ctgtaccagggcctgagcaccgccaccaaggatacctatgatgc | |
| | | cctgcacatgcaggccctgcctccaagataa | |
| | CD3 zeta | agagtgaagttcagcaggagcgcagacgcccccgcgtacaagca | 24 |
| | intracellular | gggccagaaccagctctataacgagctcaatctaggacgaagag | |
| | signaling | aggagtacgatgttttggacaagagacgtggccgggaccctgag | |
| | domain | atggggggaaagccgagaaggaagaaccctcaggaaggcctgta | |
| | (mutant) | caatgaactgcagaaagataagatggcggaggcctacagtgaga | |
| | | ttgggatgaaaggcgagcgccggaggggcaaggggcacgatggc | |
| | | ctttaccagggtctcagtacagccaccaaggacacctacgacgc | |
| | | ccttcacatgcaggccctgccccctcgctaa | |
| | 4-1BB | aagcggggcagaaagaagctgctgtacatcttcaagcagccctt | 25 |
| | CO- | catgcggcccgtgcagaccacacaagaggaagatggctgctcct | |
| | stimulatory | gcagattccccgaggaagaagaaggcggctgcgagctg | |
| | domain | aaacggggcagaaagaaactcctgtatatattcaaacaaccatt | 26 |
| | | tatgagaccagtacaaactactcaagaggaagatggctgtagct | |
| | | gccgatttccagaagaagaagaaggaggatgtgaactg | |

### EXAMPLE 1-3. Production of Lentivirus for CAR Expression

Each lentiviral plasmid was transfected into a 293T cell line (ATCC) along with three packaging DNAs (pMD.2G, pMDLg/pRRE, pRSV-rev) using Lipofectamin 3000 (Invitrogen). The culture supernatant containing the lentivirus secreted for 24-48 hours was harvested and filtered (0.45 μm filter) to remove cell residual particles, concentrated 100-fold using ultracentrifugation, and used as lentivirus concentrate for CAR-T cell production.

### EXAMPLES 1-4. Fabrication of CAR-T Cells

Leukocytes obtained by leukapheresis from normal individuals were transfected with TransAct reagent (10 μg/mL, Miltenyi) and incubated in medium containing human IL-7 (12.5 ng/mL, Miltenyi) and human IL-15 (12.5 ng/ml, Miltenyi) for 24 hours to activate T cells. Activated T cells were washed twice, transfected with lentivirus concentrate, and incubated in medium containing human IL-7 and human IL-15 for 2 days for lentivirus transduction. The transduced T cells were washed twice, transferred to fresh medium containing human IL-7 and human IL-15, and proliferated for 9 days with medium changes every 2-3 days to be used as CAR-T cells. The expression of CAR proteins on the cell surface was measured by flow cytometry (FACS-Canto II, BD Biosciences) after staining the final proliferated CAR-T cells with biotin-labeled anti-FMC63 antibody (Acrobiosystems) and PE-labeled streptavidin (BD Biosciences).

### EXAMPLES 1-5. Fabrication of Luciferase Expressed Raji Cells (Raji-Luc)

In order to artificially express Luciferase in cells, lentiviral vectors that can simultaneously express luciferase and GFP were prepared. The PLECE3-Luc vector was produced by cloning firefly luciferase ORF cDNA extracted by cleavage from pGL3-basic plasmid (Promega) at the polyenzyme cleavage site of biscistronic lentiviral vector (pLECE3), which has a multi-cloning site under the EF1a promoter and at the same time has GFP cloned under the CMV promoter (Lee SH, et al., PLOS One. 2020; 15 (1): e0223814). The pLECE3-luc plasmid along with three lentiviral packaging plasmids (pMDLg/pRRE, pRSVrev, pMD.G) was transfected into a lentivirus packaging cell line (293 FT cells, Invitrogen) using Lipofectamin 2000 reagent. After 24-48 hours, the culture supernatant containing secreted lentivirus was harvested and concentrated 10-fold using a centrifuge-type filter device. Lentivirus concentrate was added to Raji cells and transfected by centrifugation at 2500 rpm, 90 minutes at room temperature in the presence of polybrene (6 μg/ml, Sigma-Aldrich). Among the transfected Raji cells, GFP-positive cells were isolated and purified by flow cytometry (FACS-Aria II, BD Biosciences) and used as Raji-Luc cells.

### EXAMPLE 1-6. Determination of Tumor Killing and IFN-γ Secretory Capacity of CAR-T Cells

CAR-T cells (1.2×10³˜7.5×10⁵ cells/100 μl/well) proliferated for 9 days after lentiviral transduction were added to Raji-Luc cells (3×10⁴cells/50 μl/well) at various ratios (0.2-25:1) and co-cultured overnight in 96 well plates, followed by addition of 50 μl of D-Luciferin (600 μg/ml, Promega) and incubation at 37° C. for 10 min to trigger luciferase enzymatic activity in the surviving Raji-Luc cells. The luminescence of these cells was measured using a luminometer (Tecan), and the tumor cell survival rate was calculated by comparing the luminescence of untreated Raji-Luc cells to that of the CAR-T cells to determine the tumor killing capacity of the CAR-T cells. To measure the activation of CAR-T cells, CAR-T cells and Raji cells were mixed in equal numbers (3×10⁴ cells) and co-cultured in 96 well plates for 24 hours, and then the culture supernatant was harvested. The amount of IFN-γ secreted into the supernatant was measured by ELISA (human IFN-γ ELISA kit, BD Biosciences).

### EXAMPLE 1-7. Activation Marker Analysis of CAR-T Cells

To compare the degree of activation of each CAR-T cell, CAR-T cells (1×10⁵ cells/200 μl/well) proliferated for 9 days after lentiviral transduction were mixed with Raji cells (2×10⁴ cells/200 μl/well) whose proliferation was inhibited by irradiation (2000 rad) and co-cultured in 96 well plates for 3 days. During co-culture, cells were harvested every 24 hours to stain the cell surface with anti-CD69 antibody (FN50, BD Horizon), anti-CD44 antibody (IM7, Invitrogen), anti-CD25 antibody (M-A251, BioLegend), anti-CD4 antibody (RPA-T4, BD Pharmigen), anti-CD8 antibody (RPA-T8, BD Pharmigen) and anti-FMC63 scFv antibody (Y45, ACROBiosystems) to determine fluorescence intensity by flow cytometry (FACS-LSRII, BD Bioscience).

### EXAMPLE 1-8. In Vivo Efficacy Evaluation of CAR-T Cells

Immunodeficient NSG mice were injected intravenously with Raji-Luc cells (5×10⁵ cells per mouse), and after 7 days, were injected intravenously with CAR-T cells (1×10⁶ cells per mouse) proliferated for 9 days after lentiviral transfection. Thereafter, changes in tumor burden were monitored by periodic intraperitoneal injections of D-Luciferin (2 mg per mouse, Promega) followed by in vivo luminescence measurements using bioluminescence imaging equipment (IVIS, Perkin Elmer).

### EXAMPLE 2. Production and Activity Analysis of CD99L2 Backbone CAR-T Cells

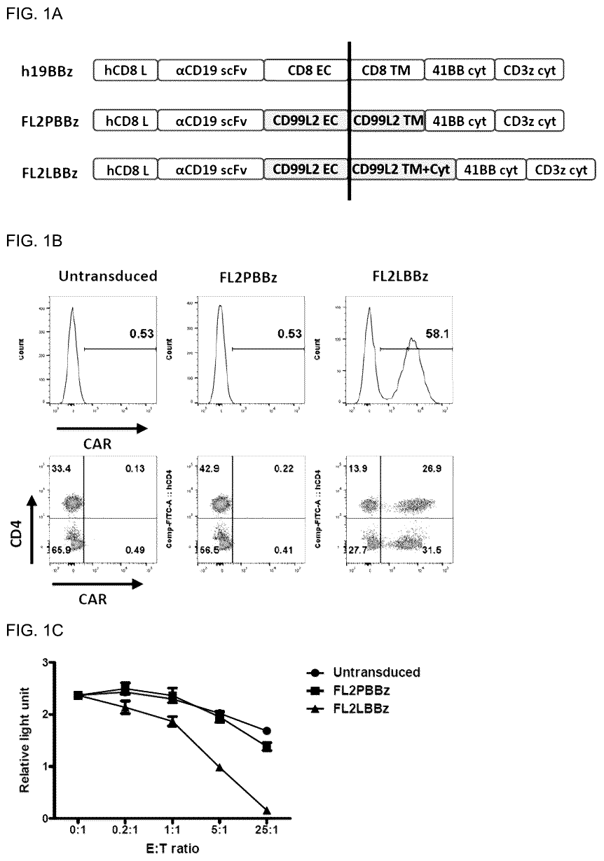

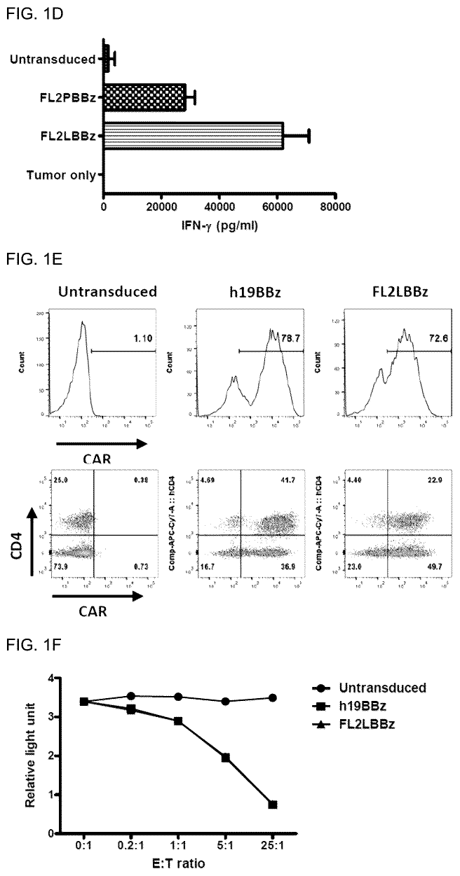

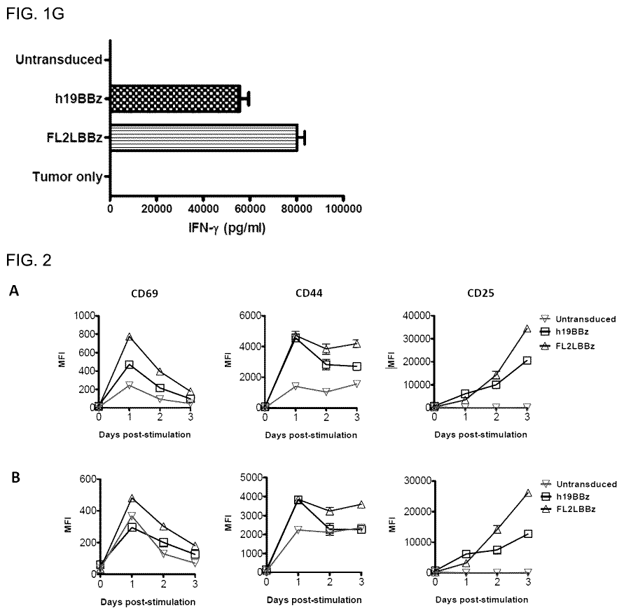

A CAR protein was produced in which the CD8 extracellular and transmembrane domains of the human CD19-targeting CD8 backbone CAR were replaced with portions of CD99L2. As the CD99L2 protein site, a construct was produced using some extracellular domain and transmembrane domain of CD99L2 (FL2PBBz), or additionally using intracellular domain (FL2LBBz) (FIG. 1A). Lentiviruses carrying the cDNAs of these CD19-targeting CD99L2 backbone CARs were constructed and then transduced into T cells isolated from human peripheral blood to generate respective CAR-T cells. The expression of CAR proteins in these CAR-T cells was measured by flow cytometry. The expression rate of FL2LBBz CAR protein was significantly higher than that of FL2PBBz in both CD4 T cells and CD8 T cells (CD4-negative T cells in the subpanel of FIG. 1B) (FIG. 1B).

Subsequently, in order to evaluate the tumor killing ability of these CAR-T cells, the CAR-T cells were co-cultured with Raji cells, which are human CD19 positive lymphoma cells. As a result, it was found that the tumor killing capacity of FL2LBBz CAR-T cells was superior to that of FL2PBBz CAR-T cells (FIG. 1C). In line with this, the amount of IFN-γ secreted by activation of CAR-T cells during co-culture with tumor cells was measured, and it was confirmed that the amount of IFN-γ secretion of FL2LBBz CAR-T cells was much higher than that of FL2PBBz CAR-T cells (FIG. 1D). Therefore, FL2LBBz CAR-T cells were selected as CD99L2 backbone CAR for future study.

To compare the in vitro tumor killing capacity and IFN-γ production capacity of FL2LBBz CAR-T cells with conventional CD8 backbone CAR-T cells (h19BBz), two CAR-T cells were produced. It was found that the mean fluorescence intensity of CAR expression per cell was slightly lower in FL2LBBz CARs compared to h19BBz CARs (FIG. 1E). However, the killing capacity against tumor cells was similar for both CAR-T cells, and in the case of IFN-γ secretion, FL2LBBz CAR-T cells showed some enhanced secretion compared to h19BBz (FIGS. 1F and 1G). Thus, it has been confirmed that CD99L2 backbone CAR-T cells have similar or enhanced in vitro activity to conventional CD8 backbone CAR-T cells.

### EXAMPLE 3. Activation Marker Analysis of CD99L2 Backbone CAR-T Cells

To further investigate the extent of tumor-induced activation of CD99L2 backbone CAR-T cells, the time-dependent expression of cell surface activation markers (CD69, CD44, CD25) that are increased upon T cell activation was measured by flow cytometry.

As a result, the rate of increase in the expression of CD69, CD44, and CD25 over time in CD99L2 backbone CAR-T cells was significantly higher compared to CD8 backbone CAR-T cells in both CD4 CAR-T cells (FIG. 2A) and CD8 CAR-T cells (FIG. 2B). Thus, CD99L2 backbone CAR-T cells demonstrated superior time-dependent activation after antigen stimulation compared to CD8 backbone CAR-T cells.

### EXAMPLE 4. Analysis of In Vivo Antitumor Effect of CD99L2 Backbone CAR-T Cells

To test the in vivo efficacy of the CD99L2 backbone CAR-T cells, immunodeficient mice (NSG mice) were injected intravenously with luciferase-expressing Raji lymphoma cells, followed by equal numbers of CD8 backbone CAR-T cells and CD99L2 backbone CAR-T cells on day 7, and the therapeutic efficacy of the two CAR-T cells was analyzed by bioluminescence imaging.

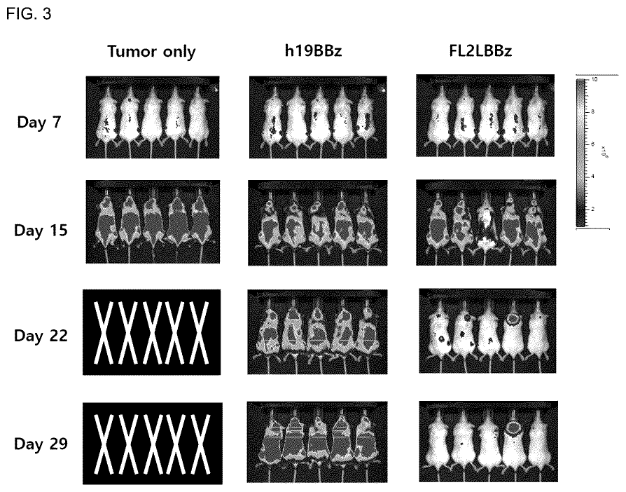

As a result, it was confirmed that CD99L2 backbone CAR-T cells showed a significant tumor removal effect at cell doses where CD8 backbone CAR-T cells showed low efficacy (FIG. 3).

From the above, CD99L2 backbone CAR-T cells have been confirmed to have significantly enhanced activation and in vivo antitumor efficacy compared to conventional CAR-T cells, suggesting the development of a novel CAR construct that gives a novel activation function to the CAR backbone.

### Industrial Applicability

According to the present invention, the T cell activation function of CD99 antigen-like 2 (CD99L2), among a cell membrane protein belonging to the CD99 family, is confirmed, and a novel chimeric antigen receptor comprising the extracellular domain of CD99L2 and the transmembrane domain of CD99L2 as a backbone is prepared. Such CD99L2-based CAR-T cells exhibit enhanced T-cell activity and tumor treatment efficiency compared to conventional backbone-based CAR-T cells, and thus can be useful in immune cell therapy for the treatment of cancer.

Although specific configurations of the present invention have been disclosed in detail, it will be obvious to those skilled in the art that the description is merely of preferable exemplary embodiments and is not to be construed as limiting the scope of the present invention. Therefore, the substantial scope of the present invention is defined by the accompanying claims and equivalents thereto.

### Sequence Listing Free Text

An electronic file is attached.

1. A chimeric antigen receptor (CAR) comprising:
(a) an antigen binding domain;
(b) a backbone comprising an extracellular spacer domain and a transmembrane domain; and
(c) an intracellular signaling domain,
wherein the extracellular spacer domain comprises a CD99L2-derived extracellular domain, and wherein the transmembrane domain comprises a CD99L2-derived transmembrane domain.

2. The chimeric antigen receptor according to claim 1, wherein the CD99L2-derived extracellular domain comprises an amino acid sequence represented by SEQ ID NO: 10.

3. The chimeric antigen receptor according to claim 1, wherein the CD99L2-derived transmembrane domain comprises an amino acid sequence represented by SEQ ID NO: 11.

4. The chimeric antigen receptor according to claim 1, wherein the chimeric antigen receptor further comprises a CD99L2-derived intracellular domain.

5. The chimeric antigen receptor according to claim 4, wherein the CD99-derived intracellular domain comprises an amino acid sequence represented by SEQ ID NO: 12.

6. The chimeric antigen receptor according to claim 1, wherein the intracellular signaling domain comprises:
an intracellular signaling domain selected from the group consisting of CD3 zeta (2), CD3 gamma (v), CD3 delta (8), CD3 epsilon (E), FcR gamma, FcR beta, CD5, CD22, CD79a, CD79b, and CD66d; and/or
a co-stimulatory domain selected from the group consisting of CD2, CD7, CD27, CD28, CD30, CD40, 4-1BB (CD137), OX40 (CD134), ICOS, LFA-1, GITR, MyD88, DAP1, PD-1, LIGHT, NKG2C, B7-H3, and a ligand specifically binding to CD83.

7. The chimeric antigen receptor according to claim 6, wherein the CD3 zeta (Z) intracellular signaling domain comprises an amino acid sequence of SEQ ID NO: 13 or 14.

8. The chimeric antigen receptor according to claim 1, wherein the antigen-binding domain comprises an antibody or antigen-binding fragment thereof that specifically binds to an antigen selected from the group consisting of:
4-1BB, BCMA, BAFF, B7-H3, B7-H6, CA9, CTAG1B, CEA, cyclin, cyclin A2, cyclin B1, CCL-1, CCR4, CD3, CD4, CD19, CD20, CD22, CD23, CD24, CD30, CD33, CD38, CD40, CD44, CD44v6, CD44v7/8, CD52, CD58, CD62, CD79A, CD79B, CD80, CD123, CD133, CD138, CD171, CSPG4, CLDN18, CLDN6, CTLA-4, c-Met, DLL3, EGFR, tEGFR, EGFRvIII, EPG-2, EPG-40, ephrin B2, EPHA2, estrogen receptor, Fc receptor, FCRL5, FGF23, FBP, FOLR1, FOLR2, GD2, ganglioside GD3, gp100, GPC3, GPCR5D, GM-CSF, Her2/neu, Her3, Her4, erbB dimer, HMW-MAA, HBsAg, HLA-A1, HLA-A2, IL-22Ra, IL-13Ra2, ICOS, IGF-1 receptor, integrin avß6, interferon receptor, IFNγR, IL-2R, IL-4R, IL-5R, IL-6R, IL-17RA, IL-31R, IL-36R, kdr, L1-CAM, CE7 epitope of L1-CAM, LRRC8A, Lewis Y, LAG3, MAGEA1, MAGEA3, MAGEA6, MAGEA10, MSLN, CMV, MUC1, NKG2D ligand, MART-1, NGF, NCAM, NRP-1, NRP-2, carcinoembryonic antigen, PD-L1, PRAME, progesterone receptor, prostate-specific antigen, PSCA, PSMA, RANKL, ROR1, SLAMF7, survivin, TPBG, TAG72, TRP1, TRP2, and Wilms' tumor 1 (WT1).

9. The chimeric antigen receptor according to claim 8, wherein the antigen-binding fragment is a single-chain variable fragment (scFv) or nanobody of an antibody.

10. The chimeric antigen receptor according to claim 1, further comprising a signal peptide at an N-terminus of the antigen-binding domain.

11. The chimeric antigen receptor according to claim 10, wherein the signal peptide is a CD8a signal peptide comprising an amino acid sequence of SEQ ID NO: 7.

12. The chimeric antigen receptor according to claim 1, wherein the chimeric antigen receptor comprises an amino acid sequence represented by SEQ ID NO: 2 or 3.

13. A nucleic acid encoding the chimeric antigen receptor according to claim 1.

14. An expression vector comprising the nucleic acid according to claim 13.

15. A virus comprising the expression vector according to claim 14.

16. An immune cell expressing the chimeric antigen receptor according to claim 1 on a surface thereof.

17. The immune cell according to claim 16, wherein the immune cell is a T cell, NK cell, NKT cell, or macrophage.

18. A composition for the treatment of cancer comprising the immune cell according to claim 16.
